(19) United States
(12) Patent Application Publication
(10) Pub. No.: US 2025/0099097 A1
(43) Pub. Date: Mar. 27, 2025

(54) Surgical Needles With Position Retainers

(71) Applicant: Curtis MATHER, Lebanon, MO (US)

(72) Inventor: Curtis MATHER, Lebanon, MO (US)

(21) Appl. No.: 18/899,490

(22) Filed: Sep. 27, 2024

Related U.S. Application Data
(60) Provisional application No. 63/540,730, filed on Sep. 27, 2023.

(51) Int. Cl. A61B 17/06; A61B 17/04

(52) U.S. Cl. CPC A61B 17/06066; A61B 17/0469

(57) ABSTRACT

A surgical needle for suturing soft tissues of a human or animal body includes a needle body terminating at a needle tip. The needle body includes at least one position retainer to inhibit unintended movement of the surgical needle during use. The position retainer may include a protuberance and/or a recess. The portion of the needle body that includes the position retainer may have a cross-sectional area that is greater or less than a cross-sectional area of one or more adjacent portions of the needle body having no position retainer.

### CROSS-REFERENCE TO RELATED APPLICATION

This application claims the benefit of U.S. Provisional Application No. 63/540,730 filed Sep. 27, 2023, the entire disclosure of which is incorporated herein by reference.

### FIELD

The present disclosure relates to surgical needles and, more particularly, surgical needles with one or more position retainers.

### BACKGROUND

This section provides background information related to the present disclosure which is not necessarily prior art.

Surgical needles are commonly used to suture soft tissues of a human or animal body and are available in a variety of sizes and configurations for penetrating various tissues. During a surgical procedure, a surgeon will insert a needle into the proper position within a tissue. However, during release or regripping of a needle driver or for other reasons, the needle may slide out of the proper position. In some cases, the needle becomes lost in the tissue, requiring the surgeon to spend time searching for the lost needle. This prolongs the surgical procedure and introduces unwanted costs and risks.

### SUMMARY

This section provides a general summary of the disclosure, and is not a comprehensive disclosure of its full scope or all of its features.

According to one aspect of the present disclosure, a surgical needle for suturing soft tissues of a body includes a needle body terminating at a needle tip. The needle body includes at least one position retainer to inhibit unintended movement of the surgical needle during use.

Additionally, at least a portion of the needle body may be tapered along its length.

Additionally, the tapered portion of the needle body may terminate at the needle tip.

Additionally, or alternatively, the at least one position retainer may include a protuberance.

Additionally, or alternatively, the needle body including the at least one position retainer may have a cross-sectional area that is greater than a cross-sectional area of one or more adjacent portions of the needle body having no position retainer.

Additionally, or alternatively, the at least one position retainer may include a recess.

Additionally, or alternatively, the needle body including the at least one position retainer may have a cross-sectional area that is less than a cross-sectional area of one or more adjacent portions of the needle body having no position retainer.

Additionally, or alternatively, the at least one position retainer may fully extend about the needle body.

Additionally, or alternatively, the at least one position retainer may extend only partially about the needle body.

Additionally, or alternatively, the at least one position retainer may lack any sharp cutting edge.

Additionally, or alternatively, the needle body may include a plurality of position retainers to inhibit unintended movement of the surgical needle during use.

Additionally, or alternatively, the at least one position retainer may have a symmetrical cross-sectional shape.

Additionally, or alternatively, the needle body and the at least one position retainer may be monolithic.

According to another aspect of the present disclosure, a method of coupling at least one position retainer to a surgical needle is disclosed. The surgical needle includes a needle body terminating at a needle tip. The at least one position retainer defines an opening therethrough. The method includes inserting the needle tip through the opening and moving the at least one position retainer to a desired location on the needle body to inhibit unintended movement of the surgical needle during use.

Additionally, the method may include using the surgical needle with the position retainer in a surgical procedure.

Additionally, or alternatively, the method may include inserting the needle tip through the opening of one or more additional position retainers.

Further areas of applicability will become apparent from the description provided herein. The description and specific examples in this summary are intended for purposes of illustration only and are not intended to limit the scope of the present disclosure.

### BRIEF DESCRIPTION OF DRAWINGS

The drawings described herein are for illustrative purposes only of selected embodiments and not all possible implementations, and are not intended to limit the scope of the present disclosure.

FIG. 1 is a half-section view of a surgical needle having a position retainer including a protuberance according to one example embodiment of the present disclosure.

FIG. 2 is a half-section view of a surgical needle having a plurality of position retainers according to another example embodiment.

FIG. 3A is a half-section view of a position retainer that can be coupled to a surgical needle.

FIG. 3B is a half-section view of a surgical needle with the position retainer of FIG. 3A coupled thereto.

FIG. 4 is a half-section view of a surgical needle having a position retainer including a recess according to yet another example embodiment.

FIG. 5 is a half-section view of a surgical needle having a two-part body including a tip portion having a position retainer according to still another example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

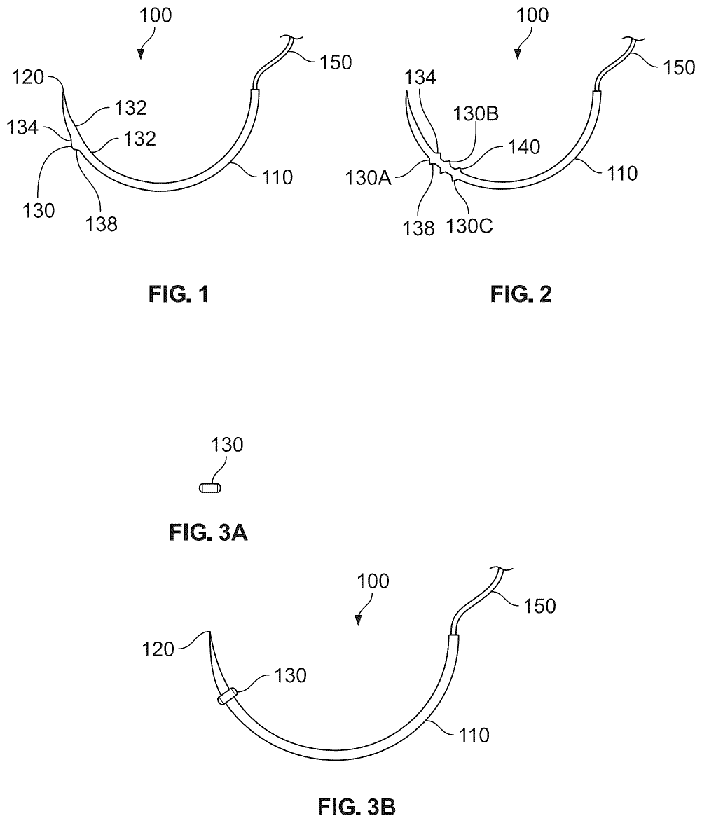

A surgical needle suitable for use in suturing soft tissues of a body according to one example embodiment of the present disclosure is illustrated in FIG. 1 and indicated generally by reference numeral 100. As shown in FIG. 1, the surgical needle 100 includes a needle body 110 terminating at a needle point 120. The needle body 110 includes a position retainer 130 to inhibit unintended movement of the surgical needle 100 during use.

In the example embodiment shown in FIG. 1, the position retainer 130 includes a protuberance. Therefore, the cross-sectional area of the needle body 110 at the location of the position retainer 130 is greater than the cross-sectional area of the needle body 110 at one or more adjacent locations 132 having no position retainer. Further, the position retainer 130 includes a ramped leading edge 134 and a ramped trailing edge 138. The ramped leading edge 134 inhibits unintended movement of the surgical needle 100 in the forward direction during a procedure, e.g., during the release and regripping of a needle driver. Similarly, the ramped trailing edge 138 inhibits unintended movement of the surgical needle 100 in the reverse direction during a procedure. In this manner, the position retainer 130 helps retain the position of the surgical needle and reduces the risk of the surgical needle 100 moving and becoming lost during a procedure.

In the particular example shown in FIG. 1, there is only one position retainer 130, the position retainer 130 protrudes from only one side of the needle body 110 (similar to a barb on a fishing hook), and the slope of the ramped trailing edge 138 is greater than the slope of the ramped leading edge 134. Further, the position retainer 130 shown in FIG. 1 has a continuous surface with no sharp edge. It should be understood, however, that the size and shape of each position retainer 130 and the number of position retainers employed can be varied as necessary for any given application of these teachings without departing from the scope of this disclosure.

In the example embodiment shown in FIG. 2, the needle body 110 includes three position retainers 130A, 130B, 130C, each including a protuberance that fully extends about the needle body 110. In this example, the position retainers 130A, 130B, 130C each have the same size and shape. In particular, each position retainer includes a ramped leading edge 134, a ramped trailing edge 138, and a discontinuous surface with a sharp edge at the intersection 140 of each leading and trailing edge. Further, the slopes of the ramped leading and trailing edges 134, 138 are the same.

In some embodiments, one or more position retainers 130 are formed monolithically with the needle body 110 as a single component. In other embodiments, the position retainers 130 may be formed separately from the needle body 110 and then attached or otherwise coupled to the needle body in any suitable manner. For example, FIG. 3A illustrates a position retainer 130 that is formed separately from the needle body 110. In this particular example, the position retainer 130 is shaped like a doughnut (i.e., a ring torus) and defines an opening through the center of the position retainer 130. The method of coupling the position retainer 130 to the surgical needle 100 includes inserting the needle point 120 through the opening and moving (e.g., sliding) the position retainer 130 to a desired location on the needle body 110. The position retainer 130 may be mechanically attached to the needle body 110 via a compression fit or in any other suitable manner, as shown in FIG. 3B.

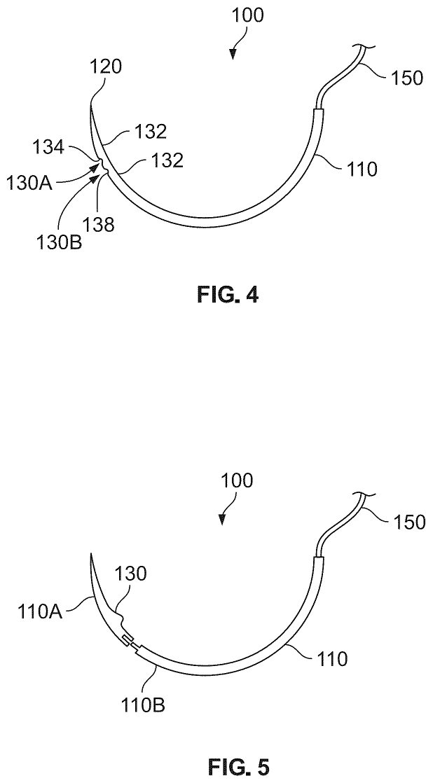

In the example embodiment shown in FIG. 4, the needle body 110 includes two position retainers 130A, 130B, each including a recess. Therefore, the cross-sectional area of the needle body 110 at the location of either position retainer 130A, 130B is less than the cross-sectional area of the needle body 110 at one or more adjacent locations 132 having no position retainer. In this example, the position retainers 130A, 130B each have the same size and shape. In particular, each position retainer includes a ramped leading edge 134, a ramped trailing edge 138, and a continuous surface with no sharp edge joining the leading and trailing edges. The slopes of the ramped leading edges 134 are greater than the slopes of the ramped trailing edges 138. Further, the position retainer recesses are formed in only one side of the needle body 110. Alternatively, the recesses may be configured differently and, e.g., extend a greater or lesser rotational distance including fully about the needle body 110.

In the example embodiment of FIG. 5, the needle body 110 includes a tip portion 110A that can be selectively attached and detached from another portion 110B of the needle body. The tip portion 110A may include one or more positions retainers 130 as shown in FIG. 5.

As should be apparent to those skilled in the art, the surgical needle 100 may have a suture 150 coupled thereto as shown in the figures for suturing soft tissues of a body. Further, the surgical needle 100 may have any desired shape and is not limited to the curved needles shown in the figures. Additionally, the diameter of the needle body 110 may be tapered along its length, including the portion of the needle body 110 that terminates at the needle point 120.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A surgical needle for suturing soft tissues of a body, the surgical needle comprising a needle body terminating at a needle tip, the needle body including at least one position retainer to inhibit unintended movement of the surgical needle during use.

2. The surgical needle of claim 1 wherein at least a portion of the needle body is tapered along its length.

3. The surgical needle of claim 2 wherein the tapered portion of the needle body terminates at the needle tip.

4. The surgical needle of claim 1 wherein the at least one position retainer comprises a protuberance.

5. The surgical needle of claim 1 wherein the needle body including the at least one position retainer has a cross-sectional area that is greater than a cross-sectional area of an adjacent portion of the needle body having no position retainer.

6. The surgical needle of claim 1 wherein the at least one position retainer comprises a recess.

7. The surgical needle of claim 1 wherein the needle body including the at least one position retainer has a cross-sectional area that is less than a cross-sectional area of an adjacent portion of the needle body having no position retainer.

8. The surgical needle of claim 1 wherein the at least one position retainer fully extends about the needle body.

9. The surgical needle of claim 1 wherein the at least one position retainer extends only partially about the needle body.

10. The surgical needle of claim 1 wherein the at least one position retainer lacks any sharp cutting edge.

11. The surgical needle of claim 1 wherein the needle body includes a plurality of position retainers including said at least one position retainer to inhibit unintended movement of the surgical needle during use.

12. The surgical needle of claim 1 wherein the at least one position retainer has a symmetrical cross-sectional shape.

13. The surgical needle of claim 1 wherein the needle body and the at least one position retainer are monolithic.

14. A method of coupling a position retainer to a surgical needle, the surgical needle including a needle body terminating at a needle tip, the position retainer defining an opening therethrough, the method comprising inserting the needle tip through the opening and moving the position retainer to a desired location on the needle body to inhibit unintended movement of the surgical needle during use.

15. The method of claim 14, further comprising using the surgical needle with position retainer in a surgical procedure.

16. The method of claim 14, further comprising inserting the needle tip through the opening of one or more additional position retainers.
